(19) United States
(12) Patent Application Publication
(10) Pub. No.: US 2010/0093637 A1
(43) Pub. Date: Apr. 15, 2010

(54) PEPTIDES AND USE THEREOF

(76) Inventors: Thierry Oddos, Meudon (FR); Gaelle Bellemere, Rouen (FR); Otto Von Stetten, Aachen (DE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/089,300

(22) PCT Filed: Oct. 10, 2006
(86) PCT No.: PCT/EP2006/009756

§ 371 Date: Jun. 22, 2009

(30) Foreign Application Priority Data
Oct. 11, 2005 (EP) 05292121.0

(51) Int. Cl. A61K 8/64; C07K 7/06; A61Q 19/08

(52) U.S. Cl. 514/15; 530/329; 530/328; 514/17

(57) ABSTRACT

The present invention relates to peptides, compositions containing such peptides, and the use thereof. The peptides derived from C-krox binding sequences of three zinc finger domains are effective for stimulating the collagen synthesis of normal human dermal fibroblast, and thus can be used to improve the appearance of the skin.

### FIELD OF THE INVENTION

The present invention relates peptides, compositions containing such peptides, and the use thereof.

### BACKGROUND OF THE INVENTION

C-krox is a transcriptional regulator of type I collagen gene expression that preferentially expressed in skin. It has been shown that c-krox can bind to two GC-rich sequences and an additional site in the promoter of the α1(I) collagen gene and to three sites in the promoter of the α2(I) collagen gene. One of the binding sites present in both promoters is adjacent to the CCAAT box. Thus, c-krox is one transcription factor controlling the coordinated expression of the two type I collagen genes in skin (P. Galera et al., Proc. Natl. Acad. Sci. Vol. 91, 9372-76, 1994). In addition, C-Krox is also known to play a major role in type II collagen expression and the chondrocyte phenotype modulation in human cells (C. Ghayor et al., J. of Biol. Chem., Vol. 275, No. 35, 27421-38, 2000).

C-Krox is a protein that displays a negative regulating effect on the expression of collagen in human dermal fibroblasts (B. Peterkofsky et al., J. of Cellular Biochem. Vol. 73, 408-22, 1999). C-krox is present predominantly in skin and its expression is higher in adult tissue than in newborn tissue (C. Ghayor et al., 2000). Thus, inhibition of the function of this protein should result in the higher synthesis of extra-cellular matrix through the use of peptide derived from its sequence and especially the one derived from the sequence binding to the DNA.

C-krox typically contains three zinc finger domains. The binding sequences of these domains have been elucidated. Applicants have derived peptides from binding sequences that stimulate the collagen synthesis of normal human dermal fibroblast.

### SUMMARY OF THE INVENTION

In one aspect, the present invention features an isolated peptide of the formula I

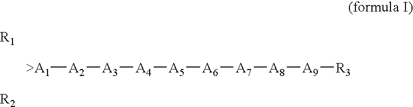

wherein formula has at least six amino acid residues; and:
A₁ is Val, Ala, Leu, Met or absent;
A₂ is Arg, Lys or absent;
A₃ is Phe, Tyr or absent;
A₄ is Thr, Ser, Ala, or Lys;
A₅ is Arg or Lys;
A₆ is Asn, Asp, Gly, or Gln;
A₇ is Asp, Asn, Glu, or absent;
A₈ is Lys, Arg or absent;
A₉ is Leu, Met, Val, Ile, Phe or absent;

provided that A3 may only be absent if A2 is absent, A2 may only be absent if A1 is absent, A7 may be absent only if A8 is absent, and A8 may only be absent if A9 is absent;

each R₁ and R₂, independently, is H, C₁₋₁₂ alkyl, C₇₋₁₀ phenylalkyl, or C(═O)E₁, where E₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E₁, the other must be H; and R₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;

or a cosmetically acceptable salt thereof.

In another aspect, the present invention relates to a composition containing a peptide of formula I and a cosmetically-acceptable carrier. In a further embodiment, the composition further contains an isolated peptide selected from the group consisting of

(a) a peptide of formula II

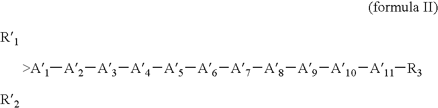

wherein:
A′₁ is Val, Ala, Leu or Met;
A′₂ is Arg or Lys;
A′₃ is Phe or Tyr;
A′₄ is Leu, Met, Val, Ile or Phe;
A′₅ is His, Tyr or Phe;
A′₆ is Ser, Thr, Ala or Lys;
A′₇ is Tyr or Phe;
A′₈ is Asp, Asn or Glu;
A′₉ is Leu, Met, Val, Ile or Phe;
A′₁₀ is Lys or Arg;
A′₁₁ is Asn, Asp, Gly or Gln;

each R′₁ and R′₂, independently, is H, C₁₋₁₂ alkyl, C₇₋₁₀ phenylalkyl, or C(═O)E₁, where E′₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E′₁, the other must be H; and

R′₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;

or a cosmetically-acceptable salt thereof;

(b) a peptide of Formula III,

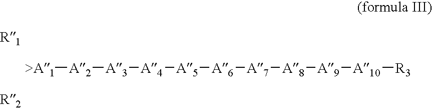

wherein:
A″₁ is Cys or Ser;
A″₂ is His, Tyr or Phe;
A″₃ is Lys or Arg;
A″₄ is Leu, Met, Val, Ile or Phe;
A″₅ is Leu, Met, Val, Ile or Phe;
A″₆ is His, Tyr or Phe;
A″₇ is Asn, Asp, Gly or Gln;
A″₈ is Val, Ala, Leu or Met;
A″₉ is Asn, Asp, Gly or Gln;
A″₁₀ is Lys or Arg;

each R″₁ and R″₂, independently, is H, C₁₋₁₂ alkyl, C₇₋₁₀ phenylalkyl, or C(═O)E″₁, where E₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E″₁, the other must be H; and

R″₃ is OH, NH₂, C₁₋₁₂ alkoxy, C₇₋₁₀ phenylalkoxy, C₁₁₋₁₅ naphthylalkoxy, C₁₋₁₂ alkylamino, C₇₋₁₀ phenylalkylamino, or C₁₁₋₁₄ naphthylalkylamino;

or a cosmetically-acceptable salt thereof; or

(c) a fragment of a peptide of either formula II or formula III, wherein such fragment contains at least six amino acid residues.

Other features and advantages of the present invention will be apparent from the detailed description of the invention and from the claims.

### DETAILED DESCRIPTION OF THE INVENTION

It is believed that one skilled in the art can, based upon the description herein, utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### DEFINITIONS

What is meant by a “product” is a product in finished packaged form. In one embodiment, the package is a container such as a plastic, metal or glass tube or jar containing the composition. The product may further contain additional packaging such as a plastic or cardboard box for storing such container.

In one embodiment, the product contains instructions directing the user to administer the composition to the skin to treat at least one sign of aging on the skin selected from the group consisting of enhancing the elasticity of the skin, enhancing the firmness of the skin, smoothing the surface of the skin, and reducing the appearance of wrinkles on the skin.

What is meant by “enhancing the elasticity” of the skin is enhancing the elasticity or preventing the loss of elasticity of the skin.

What is meant by “enhancing the firmness” of the skin is enhancing the firmness or preventing the loss of firmness of the skin.

As used herein, the term “wrinkle” includes fine line, fine wrinkles, coarse wrinkles, cellulite, scars, and stretch marks. Examples of wrinkles include, but are not limited to, fine lines around the eyes (e.g., “crow's feet”), forehead and cheek wrinkles, frown-lines, and laugh-lines around the mouth.

What is meant by “promoting” is promoting, advertising, or marketing. Examples of promoting include, but are not limited to, written, visual, or verbal statements made on the product or in stores, magazines, newspaper, radio, television, internet, and the like.

For promoting the treatment of signs of aging, examples of such statements include, but are not limited to, “enhances skin elasticity,” “helps reduce the appearance of photodamaged skin,” “improving visible and tactilely perceptible manifestations of the skin,” “increases skin elasticity or firmness,” “restores skin elasticity,” “treats sagging or lax skin,” “reduces the appearance of cellulite,” “lifts the skin,” “younger looking skin,” “smoothing under eye bags,” “smoothing skin texture,” “lifts the face,” “younger skin,” and “makes skin look younger.”

As used herein, “administering to skin in need of such treatment” means contacting (e.g., by use of the hands or an applicator such, but not limited to, a wipe, tube, roller, spray, or patch) the area of skin in need such treatment. These features may be present on the face such as under or adjacent the eyes, or on the forehead, cheeks, jowls, and neck as well as other areas of the body such as the arms and legs (e.g., cellulite).

As used herein, “composition” means a composition suitable for administration to the skin.

As used herein, “cosmetically-acceptable” means that the ingredients or compositions which the term describes are suitable for use in contact with the skin without undue toxicity, incompatibility, instability, irritation, allergic response, and the like. This term is not intended to limit the ingredient/composition to which it describes for use solely as a cosmetic (e.g., the ingredient/composition may be a pharmaceutical agent).

As used herein, “safe and effective amount” means an amount of the compound, carrier, or of the composition sufficient to induce an enhancement in tissue elasticity, but low enough to avoid serious side effects. The safe and effective amount of the compounds or composition will vary with the area being treated, the age, health and skin type of the end user, the duration and nature of the treatment, the specific compound or composition employed, the particular cosmetically-acceptable carrier utilized, and like factors.

### Peptides

The composition of the present invention contains a peptide of the formula I

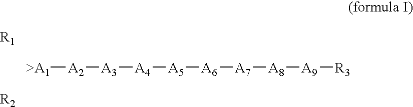

wherein formula I has at least six amino acid residues; and:

A₁ is Val, Ala, Leu, Met or absent;

A₂ is Arg, Lys or absent;

A₃ is Phe, Tyr or absent;

A₄ is Thr, Ser, Ala, or Lys;

A₅ is Arg or Lys;

A₆ is Asn, Asp, Gly, or Gln;

A₇ is Asp, Asn, Glu, or absent;

A₈ is Lys, Arg or absent;

A₉ is Leu, Met, Val, Ile, Phe or absent;

provided that A3 may only be absent if A2 is absent, A2 may only be absent if A1 is absent, A7 may be absent only if A8 is absent, and A8 may only be absent if A9 is absent;

each R₁ and R₂, independently, is H, CO₁₋₁₂ alkyl, C₇₋₁₀ phenylalkyl, or C(═O)E₁, where E₁ is C₁₋₁₂ alkyl, C₃₋₁₉ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E₁, the other must be H; and

R₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;

or a cosmetically acceptable salt thereof.

In one embodiment, R₁ and R₂, which are bound to the N-terminus of the peptide, are both H. In another embodiment, R₁ is H and R₂ is C(═O)E₁ (e.g., palmitoyl, oleatoyl, or stearatoyl).

Examples of peptides of the present invention include, but are not limited to H₂-Val-Arg-Phe-Thr-Arg-Asn-Asp-Lys-Leu-NH₂, H₂-Val-Arg-Phe-Thr-Arg-Asn-Asp-Lys-Leu-OH, H₂-Val-Arg-Phe-Thr-Arg-Asn-NH₂, H₂-Val-Arg-Phe-Thr-Arg-Asn-OH (“Peptide 1”), H₂-Thr-Arg-Asn-Asp-Lys-Leu-NH₂, H₂-Thr-Arg-Asn-Asp-Lys-Leu-OH (“Peptide 2”), Palmitoyl-Val-Arg-Phe-Thr-Arg-Asn-Asp-Lys-Leu-NH₂, Palmitoyl-Val-Arg-Phe-Thr-Arg-Asn-Asp-Lys-Leu-OH, Palmitoyl-Val-Arg-Phe-Thr-Arg-Asn-NH₂, Palmitoyl-Val-Arg-Phe-Thr-Arg-Asn-OH, Palmitoyl-Thr-Arg-Asn-Asp-Lys-Leu-NH₂, Palmitoyl-Thr-Arg-Asn-Asp-Lys-Leu-OH, Stearatoyl-Val-Arg-Phe-Thr-Arg-Asn-Asp-Lys-Leu-NH₂, Stearatoyl-Val-Arg-Phe-Thr-Arg-Asn-Asp-Lys-Leu-OH, Stearatoyl-Val-Arg-Phe-Thr-Arg-Asn-NH₂, Stearatoyl-Val-Arg-Phe-Thr-Arg-Asn-OH, Stearatoyl-Thr-Arg-Asn-Asp-Lys-Leu-NH₂, Stearatoyl-Thr-Arg-Asn-Asp-Lys-Leu-OH, or a cosmetically-acceptable salt thereof.

In another aspect, the composition of the present invention may further contain one or more isolated peptides selected from the group consisting of

(a) a peptide of formula II

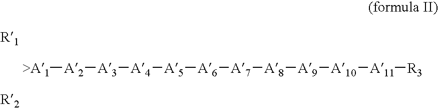

wherein:
A′₁ is Val, Ala, Leu or Met;
A′₂ is Arg or Lys;
A′₃ is Phe or Tyr;
A′₄ is Leu, Met, Val, Ile or Phe;
A′₅ is His, Tyr or Phe;
A′₆ is Ser, Thr, Ala or Lys;
A′₇ is Tyr or Phe;
A′₈ is Asp, Asn or Glu;
A′₉ is Leu, Met, Val, Ile or Phe;
A′₁₀ is Lys or Arg;
A′₁₁ is Asn, Asp, Gly or Gln;

each R′₁ and R′₂, independently, is H, C₁₋₁₂ alkyl, C₇₋₁₀ phenylalkyl, or C(═O)E₁, where E′₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E′₁, the other must be H; and

R′₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;

or a cosmetically-acceptable salt thereof;

(b) a peptide of Formula III,

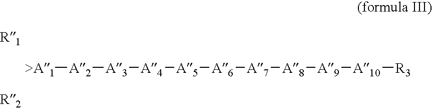

wherein:
A″₁ is Cys or Ser;
A″₂ is His, Tyr or Phe;
A″₃ is Lys or Arg;
A″₄ is Leu, Met, Val, Ile or Phe;
A″₅ is Leu, Met, Val, Ile or Phe;
A″₆ is His, Tyr or Phe;
A″₇ is Asn, Asp, Gly or Gln;
A″₈ is Val, Ala, Leu or Met;
A″₉ is Asn, Asp, Gly or Gln;
A″₁₀ is Lys or Arg;

each R″₁ and R″₂, independently, is H, C₁₋₁₂ alkyl, C₇₋₁₀ phenylalkyl, or C(═O)E″₁, where E₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E″₁, the other must be H; and

R″₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;

or a cosmetically-acceptable salt thereof; or

(c) a fragment of a peptide of either formula II or formula III, wherein the fragment includes at least six amino acid residues. Examples of such fragments include peptides for the formula

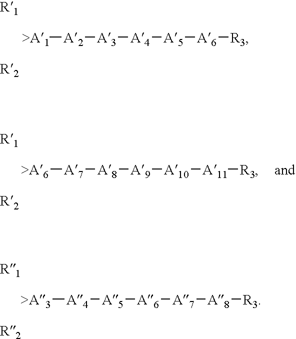

The symbol A₁, A₂, or the like used herein (e.g., in FIG. 1) stands for the residue of an alpha-amino acid. Such symbols represent the general structure, —NH—CH(X)—CO— or ═N—CH(X)—CO— when it is at the N-terminus or —NH—CH(X)—CO— when it is not at the N-terminus, where X denotes the side chain (or identifying group) of the alpha-amino acid, e.g., X is —CH(CH₃)₂ for Val. Note that the N-terminus is at the left and the C-terminus at the right in accordance with the conventional representation of a polypeptide chain. R₁ and R₂ are both bound to the free nitrogen atom N-terminal amino acid (e.g., A₁ or A₂) and the R₃ is bound to the free carboxyl group of the C-terminal amino acid (e.g., A₅ or A₆).

Furthermore, where the amino acid residue is optically active, it is the L-form configuration that is intended unless the D-form is expressly designated. An alkyl group, if not specified, contains 1-12 carbon atoms.

The peptide of the invention can be provided in the form of cosmetically acceptable salts. Examples of preferred salts are those with therapeutically acceptable organic acids, e.g., acetic, palmitic, oleic, stearic, lactic, maleic, citric, malic, ascorbic, succinic, benzoic, salicylic, methanesulfonic, or pamoic acid, as well as polymeric acids such as tannic acid or carboxymethyl cellulose, and salts with inorganic acids such as the hydrohalic acids (e.g., hydrochloric acid), sulfuric acid or phosphoric acid.

The amount of peptide present in the composition will depend on the peptide used. The peptide typically will be present in the composition in an amount from about 0.001% to about 10% by weight, in particular in an amount from about 0.01% to about 1% by weight.

The method for synthesizing peptides of the present invention are well documented and are within the ability of a person of ordinary skill in the art. See, e.g., Bodanszky M, Int J Pept Protein Res 25(5):449-74 (1985), Fmoc Solid Phase Peptide Synthesis, eds. Chan, W. & White, P. (Oxford University Press, 2000), and Chemical Approaches to the Synthesis of Peptides and Proteins, Lloyd-Williams, P. et al. (CRC Press, 1997).

### Compositions

The compositions useful in the present invention involve formulations suitable for administering to the target tissues. In one embodiment, the composition contains a safe and effective amount of (i) the peptide and (ii) a cosmetically-acceptable carrier. In one embodiment, the cosmetically-acceptable carrier is from about 90% to about 99.99%, by weight, of the composition (e.g., from about 95% to about 99%, by weight, of the composition).

The compositions may be made into a wide variety of product types that include but are not limited to solutions, suspensions, lotions, creams, gels, sticks, sprays, ointments, cleansing liquid washes and solid bars, shampoos and hair conditioners, pastes, foams, powders, mousses, shaving creams, wipes, patches, nail lacquers, wound dressing and adhesive bandages, hydrogels, film-forming products, facial and skin masks, make-up such as foundations, mascaras, and lipsticks, liquid drops, vaginal washes, suppositories, tampons, toothpastes, mouthwashes, lozenges, tablets, gums and candy, mucoadhesives, and the like. These product types may contain several types of cosmetically-acceptable carriers including, but not limited to solutions, suspensions, emulsions such as microemulsions and nanoemulsions, gels, solids and liposomes. The following are non-limitative examples of such carriers. Other carriers can be formulated by those of ordinary skill in the art.

The compositions useful in the present invention can be formulated as solutions. Solutions typically include an aqueous or organic solvent (e.g., from about 90% to about 99% of a cosmetically acceptable aqueous or organic solvent). Examples of suitable organic solvents include: propylene glycol, polyethylene glycol (200-600), polypropylene glycol (425-2025), glycerol, 1,2,4-butanetriol, sorbitol esters, 1,2,6-hexanetriol, ethanol, and mixtures thereof.

A lotion can be made from such a solution. Lotions typically contain from about 1% to about 20% (e.g., from about 5% to about 10%) of an emollient(s) and from about 70% to about 90% (e.g., from about 75% to about 80%) of water. As used herein, “emollients” refer to materials used for the prevention or relief of dryness, as well as for the protection of the skin or hair. Examples of emollients include, but are not limited to, those set forth in the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”).

Another type of product that may be formulated from a solution is a cream. A cream typically contains from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may contain a simple base of animal, vegetable, or synthetic oils or semi-solid hydrocarbons. An ointment may contain from about 2% to about 10% of an emollient(s) plus from about 0.1% to about 2% of a thickening agent(s). Examples of thickening agents include, but are not limited to, those set forth in the ICI Handbook pp. 1693-1697.

The compositions useful in the present invention can also be formulated as emulsions. If the carrier is an emulsion, from about 1% to about 10% (e.g., from about 2% to about 5%) of the carrier contains an emulsifier(s). Emulsifiers may be nonionic, anionic or cationic. Examples of emulsifiers include, but are not limited to, those set forth in the ICI Handbook, pp. 1673-1686.

Lotions and creams can be formulated as emulsions. Typically such lotions contain from 0.5% to about 5% of an emulsifier(s), while such creams would typically contain from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the art and are useful in the subject invention. Multiphase emulsion compositions, such as the water-in-oil-in-water type or the oil-in-water-in-oil type, are also useful in the subject invention. In general, such single or multiphase emulsions contain water, emollients, and emulsifiers as essential ingredients.

The compositions of this invention can also be formulated as a gel (e.g., an aqueous, alcohol, alcohol/water, or oil gel using a suitable gelling agent(s)). Suitable gelling agents for aqueous and/or alcoholic gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically contains between about 0.1% and 5%, by weight, of such gelling agents.

The compositions of the present invention can also be formulated into a solid formulation (e.g., a wax-based stick, soap bar composition, powder, wipe containing powder, lozenge, suppository, candy, or gum).

The compositions useful in the subject invention may contain, in addition to the aforementioned components, a wide variety of additional oil-soluble materials and/or water-soluble materials conventionally used in compositions for use on skin and mucosal tissues at their art-established levels.

### Additional Cosmetically Active Agents

In one embodiment, the composition further contains another cosmetically active agent in addition to the peptide. What is meant by a “cosmetically active agent” is a compound (e.g., a synthetic compound or a compound isolated from a natural source, or a natural extract containing a mixture of compounds) that has a cosmetic or therapeutic effect on the tissue, including, but not limiting to, lightening agents, darkening agents such as self-tanning agents, anti-acne agents, shine control agents, anti-microbial agents such as anti-yeast agents, anti-fungal, and anti-bacterial agents, anti-inflammatory agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, hair growth enhancing agents, hair growth delaying agents, firming agents, anti-callous agents, agents for skin conditioning, anti-cellulite agents, fluorides, teeth whitening agents, anti-plaque agents, and plaque-dissolving agents, and odor-control agents such as odor masking or pH-changing agents.

In one embodiment, the agent is selected from, but not limited to, the group consisting of hydroxy acids, benzoyl peroxide, D-panthenol, octyl methoxycinnimate, titanium dioxide, octyl salicylate, homosalate, avobenzone, carotenoids, free radical scavengers, spin traps, retinoids and retinoid precursors such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, amino acids such a proline, vitamins, lactobionic acid, acetyl-coenzyme A, niacin, riboflavin, thiamin, ribose, electron transporters such as NADH and FADH2, and other botanical extracts such as aloe vera, Feverfew, and Soy, and derivatives and mixtures thereof. The cosmetically active agent will typically be present in the composition of the invention in an amount of from about 0.001% to about 20% by weight of the composition, e.g., about 0.005% to about 10% such as about 0.01% to about 5%.

Examples of vitamins include, but are not limited to, vitamin A, vitamin Bs such as vitamin B3, vitamin B5, and vitamin B12, vitamin C, vitamin K, vitamin E such as alpha, gamma or delta-tocopherol, and derivatives and mixtures thereof.

Examples of hydroxy acids include, but are not limited, to glycolic acid, lactic acid, malic acid, salicylic acid, citric acid, and tartaric acid.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), different types of tocopherols (e.g., alpha-, gamma-, and delta-tocopherols and their esters such as acetate) and their mixtures, tocotrienols, and ubiquinone. Natural extracts containing antioxidants suitable for use in the compositions of this invention, include, but not limited to, extracts containing flavonoids, isoflavonoids, and their derivatives such as genistein and diadzein (e.g., such as Soy and Clover extracts, extracts containing resveratrol and the like. Examples of such natural extracts include grape seed, green tea, pine bark, and propolis.

### Other Materials

Various other materials may also be present in the compositions useful in the subject invention. These include humectants, proteins and polypeptides, preservatives and an alkaline agent. Examples of such agents are disclosed in the ICI Handbook, pp. 1650-1667. The compositions of the present invention may also contain chelating agents (e.g., EDTA) and preservatives (e.g., parabens). Examples of suitable preservatives and chelating agents are listed in pp. 1626 and 1654-55 of the ICI Handbook. In addition, the compositions useful herein can contain conventional cosmetic adjuvants, such as colorants such as dyes and pigments, opacifiers (e.g., titanium dioxide), and fragrances.

### Mineral Water

The compositions of the present invention may be prepared using a mineral water, for example mineral water that has been naturally mineralized such as Evian® Mineral Water (Evian, France). In one embodiment, the mineral water has a mineralization of at least about 200 mg/L (e.g., from about 300 mg/L to about 1000 mg/L). In one embodiment, the mineral water contains at least about 10 mg/L of calcium and/or at least about 5 mg/L of magnesium.

The composition and formulations containing such compositions of the present invention may be prepared using methodology that is well known by an artisan of ordinary skill.

### Use

The peptides and compositions of the present invention may be topically applied to the skin. In one embodiment, the invention features a method of treating at least one sign of aging on the skin selected from the group consisting of enhancing the elasticity of the skin, enhancing the firmness of the skin, smoothing the surface of the skin, and reducing the appearance of wrinkles on the skin wherein the method includes applying to skin in need of such treatment at least one peptide of formula I, II, or III or composition containing such peptide(s).

### Example 1

The stimulation of collagen synthesis by peptides of the present invention was evaluated by measuring the incorporation of tritiated proline in fibroblasts collagenic fractions. The assay was conducted as follows. Human fibroblasts (obtained from the human biopsy of a 42 year old) were seeded in 24-well plates at approximately 40,000 cells per well in 1 ml of culture medium. The culture medium contained DMEM (Invitrogen S.a.r.l., Cergy Pontoise, France) supplemented with 10% Fetal Calf Serum (FCS, Cambrex Bio Science Paris S.a.r.l., Emerainville, France), 1% Glutamax (Invitrogen S.a.r.l.), 4 μg/ml gentamicine (Invitrogen S.a.r.l.), and 2.5 μg/ml Fungizone (Invitrogen S.a.r.l.). Cells were placed at 37° C. under 5% CO₂ in a humidified atmosphere for 72 hours.

The culture medium was the removed and replaced with 1 ml of either: (i) incubation medium alone (untreated control); (ii) incubation medium further containing 1 ng/ml TGFb1 (R&D Systems Europe, Lille, France) for use as a stimulated positive control; or (iii) incubation medium further containing one of the tested peptides at a concentration of 1 μg/ml or 10 μg/ml. The incubation medium contained DMEM supplemented with 2% FCS, 1% Glutamax, 4 μg/ml gentamicine, 2.5 μg/ml Fungizone, 50 μg/ml ascorbic acid, 50 μg/ml b-aminoproionitrile, and 3.4 μg/ml a ketoglutarate.

During the last 24 hours of incubation, five μCi of tritiated proline (L-2,3-3H proline, Amersham Biosciences Europe, Saclay, France) was also added to each well. Each condition (untreated control, positive control and tested peptide) was performed in triplicate. In order to normalize the results, the same experiment was performed in parallel, except that tritiated proline was omitted. At the end of the treatment, cells were counted with a coulter counter. After 72 hours of treatment, incubation medium (supernatants) were collected in 2 ml microtubes and frozen at −20° C.

In order to perform the collagen extraction, 1 ml of cold 20% TCA (Sigma-Aldrich, Lyon, France) was added to 1 ml of supernatant. The samples were then vortexed, incubated for 5 minutes (min) at −20° C., and let stand on ice for 30 min. The samples were then centrifuged (2300 g, 5 min, 4° C.). The supernatants were then eliminated, and the pellets were rinsed with 1 ml of cold 5% TCA, vortexed, and centrifuged (2300 g, 5 min, 4° C.). The pellets were then rinsed twice with 5% TCA following the same procedure as described above. The pellets were then dissolved in 0.3 ml of 0.05 N NaOH and neutralized with 0.1N HCl to reach pH 6-8. Then, 0.2 ml of protein solution was incubated for 2 hours at 37° C. in 2×Tris HCl Buffer containing 10 units of collagenase activity (Clostridium hystolyticum Type III collagenase, Sigma-Aldrich, Lyon, France).

The enzymatic reaction was then stopped by precipitation with 500 μl of 15% TCA and 500 μl of 0.75% tannic acid. Samples were then incubated 30 min on ice. The precipitates were then centrifuged (2300 g, 5 min, 4° C.). The supernatants containing collagen fractions were collected, and scintillation liquid (Ecolume ICN) was added to each sample (5 ml to 100 μl sample). Then, radioactivity was measured in a β-scintillation counter.

The rate of collagen synthesis was calculated as follows: Synthesis rate=(number of dpm/cell number)*1000

The percentage of stimulation was calculated as follows: % of stimulation=[(synthesis rate in sample-synthesis rate in control)/(synthesis rate in control)]*100.

The results are depicted in Table 1.

TABLE 1

| | Percent stimulation |
|---|---|
| Incubation Medium | vs. control |
| TGF b (1 ng/ml) | 45.7 |
| Peptide 1 (1 ug/ml) | 14.8 |
| Peptide 1 (10 ug/ml) | 43.1 |
| Peptide 2 (1 ug/ml) | 23.5 |
| Peptide 2 (10 ug/ml) | 42.6 |

The results show that the peptides VFTRN and TRNDKL were able to markedly stimulate collagen synthesis at 10 μg/ml for both peptides by 43% and 42% respectively, a level of stimulation similar to the one of TGFb (+45%) a potent stimulator of collagen synthesis in dermal fibroblast used as positive control. At lower concentration (1 μg/ml) both peptide stimulate also collagen synthesis but to a lower extent (+15% and +25% respectively).

1. An isolated peptide of the formula I 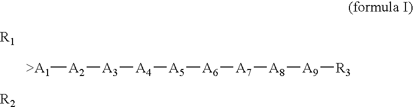
wherein formula I has at least six amino acid residues; and:
A₁ is Val, Ala, Leu, Met or absent;
A₂ is Arg, Lys or absent;
A₃ is Phe, Tyr or absent;
A₄ is Thr, Ser, Ala, or Lys;
A₅ is Arg or Lys;
A₆ is Asn, Asp, Gly, or Gln;
A₇ is Asp, Asn, Glu, or absent;
A₈ is Lys, Arg or absent;
A₉ is Leu, Met, Val, Ile, Phe or absent;
provided that A3 may only be absent if A2 is absent, A2 may only be absent if A1 is absent, A7 may be absent only if A8 is absent, and A8 may only be absent if A9 is absent;
each R₁ and R₂, independently, is H, C₁₋₁₂ alkyl, C₇₋₁₀ phenylalkyl, or C(═O)E₁, where E₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E₁, the other must be H; and
R₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;
or a cosmetically acceptable salt thereof.

2. An isolated peptide of claim 1, wherein R₁ is H, R₂ is H or C(═O)E₁ where E₁ is C₁₋₁₂ alkyl, and R₃ is OH or NH₂.

3. An isolated peptide of claim 1, wherein A₁ is Val; A₂ is Arg; A₃ is Phe; A₄ is Thr; A₅ is Arg; A₆ is Asn; A₇ is Asp or absent; A₈ is Lys or absent; and A₉ is Leu or absent.

4. An isolated peptide of claim 1, wherein A₁ is Val; A₂ is Arg; A₃ is Phe; A₄ is Thr; A₅ is Arg; A₆ is Asn; A₇ is absent; A₈ is absent; and A₉ is absent.

5. An isolated peptide of claim 1, wherein A₁ is Val or absent; A₂ is Arg or absent; A₃ is Phe or absent; A₄ is Thr; A₅ is Arg; A₆ is Asn; A₇ is Asp; A₈ is Lys; and A₉ is Leu.

6. An isolated peptide of claim 1, wherein A₁ is absent; A₂ is absent; A₃ is absent; A₄ is Thr; A₅ is Arg; A₆ is Asn; A₇ is Asp; A₈ is Lys; and A₉ is Leu.

7. An isolated peptide of claim 1, wherein A₁ is Val; A₂ is Arg; A₃ is Phe; A₄ is Thr; A₅ is Arg; A₆ is Asn; A₇ is Asp; A₈ is Lys; and A₉ is Leu.

8. An isolated peptide of any of the preceding claims, wherein R₁ is H, R₂ is H or C(═O)E₁ where E₁ is C₁₋₁₂ alkyl, and R₃ is OH or NH₂.

9. An isolated peptide of any of the preceding claims, wherein R₁ is H, R₂ is H or C(═O)E₁ where E₁ is C₁₋₂₀ alkyl, and R₃ is OH or NH₂.

10. An isolated peptide of claim 1, wherein said peptide is H₂-Val-Arg-Phe-Thr-Arg-Asn-Asp-Lys-Leu-NH₂, H₂-Val-Arg-Phe-Thr-Arg-Asn-Asp-Lys-Leu-OH, H₂-Val-Arg-Phe-Thr-Arg-Asn-NH₂, H₂-Val-Arg-Phe-Thr-Arg-Asn-OH, H₂-Thr-Arg-Asn-Asp-Lys-Leu-NH₂, H₂-Thr-Arg-Asn-Asp-Lys-Leu-OH, Palmitoyl-Val-Arg-Phe-Thr-Arg-Asn-Asp-Lys-Leu-NH₂, Palmitoyl-Val-Arg-Phe-Thr-Arg-Asn-Asp-Lys-Leu-OH, Palmitoyl-Val-Arg-Phe-Thr-Arg-Asn —NH₂, Palmitoyl-Val-Arg-Phe-Thr-Arg-Asn-OH, Palmitoyl-Thr-Arg-Asn-Asp-Lys-Leu-NH₂, Palmitoyl-Thr-Arg-Asn-Asp-Lys-Leu-OH, Stearatoyl-Val-Arg-Phe-Thr-Arg-Asn-Asp-Lys-Leu-NH₂, Stearatoyl-Val-Arg-Phe-Thr-Arg-Asn-Asp-Lys-Leu-OH, Stearatoyl-Val-Arg-Phe-Thr-Arg-Asn-NH₂, Stearatoyl-Val-Arg-Phe-Thr-Arg-Asn-OH, Stearatoyl-Thr-Arg-Asn-Asp-Lys-Leu-NH₂, or Stearatoyl-Thr-Arg-Asn-Asp-Lys-Leu-OH, or a cosmetically-acceptable salt thereof.

11. A composition comprising a peptide of any of the preceding claims and a cosmetically-acceptable carrier.

12. A composition of claim 12, wherein said composition further comprises one or more of the following isolated peptides selected from the group consisting of
(a) a peptide of formula II 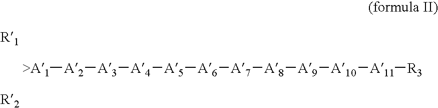
wherein:
A′₁ is Val, Ala, Leu or Met;
A′₂ is Arg or Lys;
A′₃ is Phe or Tyr;
A′₄ is Leu, Met, Val, Ile or Phe;
A′₅ is His, Tyr or Phe;
A′₆ is Ser, Thr, Ala or Lys;
A′₇ is Tyr or Phe;
A′₈ is Asp, Asn or Glu;
A′₉ is Leu, Met, Val, Ile or Phe;
A′₁₀ is Lys or Arg;
A′₁₁ is Asn, Asp, Gly or Gln;
each R′₁ and R′₂, independently, is H, C₁₋₁₂ alkyl, C₇₋₁₀ phenylalkyl, or C(═O) E₁, where E′₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E′₁, the other must be H; and
R′ₛ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₄ naphthylalkylamino;
or a cosmetically-acceptable salt thereof.
(b) a peptide of Formula III, 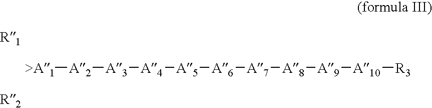
wherein:
A″₁ is Cys or Ser;
A″₂ is His, Tyr or Phe;
A″₃ is Lys or Arg;
A″₄ is Leu, Met, Val, Ile or Phe;
A″₅ is Leu, Met, Val, Ile or Phe;
A″₆ is His, Tyr or Phe;
A″₇ is Asn, Asp, Gly or Gln;
A″₈ is Val, Ala, Leu or Met;
A″₉ is Asn, Asp, Gly or Gln;
A″₁₀ is Lys or Arg;
each R″₁ and R″₂, independently, is H, C₁₋₁₂ alkyl, C₇₋₁₀ phenylalkyl, or C(═O)E″₁, where E₁ is C₁₋₁₂ alkyl, C₃₋₁₄ alkenyl, C₃₋₁₄ alkynyl, phenyl, 3,4-dihydroxyphenylalkyl, naphthyl, or C₇₋₁₀ phenylalkyl; provided that when either R₁ or R₂ is C(═O)E″₁, the other must be H; and
R″₃ is OH, NH₂, C₁₋₁₂ alkoxy, C₇₋₁₀ phenylalkoxy, C₁₁₋₁₄ naphthylalkoxy, C₁₋₁₂ alkylamino, C₇₋₁₀ phenylalkylamino, or C₁₁₋₁₉ naphthylalkylamino;
or a cosmetically-acceptable salt thereof; or
(c) a fragment of a peptide of either formula II or formula III, wherein said fragment comprises at least six amino acid residues.

13. A method of treating at least one sign of aging on the skin selected from the group consisting of enhancing the elasticity of said skin, enhancing the firmness of said skin, smoothing the surface of the skin, and reducing the appearance of wrinkles on the skin, wherein the method comprises applying to skin in need of such treatment a composition of claim 12 or 13.


SEQUENCE LISTING (9 sequences)

SEQ ID NO: 1 - protein, 9 aa, Homo sapiens
VRFTRNDKL

SEQ ID NO: 2 - protein, 6 aa, Homo sapiens
VRFTRN

SEQ ID NO: 3 - protein, 6 aa, Homo sapiens
TRNDKL

SEQ ID NO: 4 - protein, 9 aa, Homo sapiens
VRFTRNDKL

SEQ ID NO: 5 - protein, 6 aa, Homo sapiens
VRFTRN

SEQ ID NO: 6 - protein, 6 aa, Homo sapiens
TRNDKL

SEQ ID NO: 7 - protein, 9 aa, Homo sapiens
VRFTRNDKL

SEQ ID NO: 8 - protein, 6 aa, Homo sapiens
VRFTRN

SEQ ID NO: 9 - protein, 6 aa, Homo sapiens
TRNDKL